(19) United States
(12) Patent Application Publication
(10) Pub. No.: US 2025/0144237 A1
(43) Pub. Date: May 8, 2025

(54) AGENTS, COMPOSITIONS AND METHODS FOR ENHANCED DELIVERY OF NUCLEIC ACIDS TO THE CELLS WITHIN THE RESPIRATORY SYSTEM

(71) Applicant: Cila Therapeutics Inc., Jamaica Plain, MA (US)

(72) Inventors: Safia K. Rizvi, Jamaica Plain, MA (US); Larry Lands, Hampstead (CA)

(73) Assignee: Cila Therapeutics Inc., Jamaica Plain, MA (US)

(21) Appl. No.: 18/939,439

(22) Filed: Nov. 6, 2024

Related U.S. Application Data
(60) Provisional application No. 63/547,629, filed on Nov. 7, 2023.

(51) Int. Cl. A61K 48/00; A61K 9/00; A61K 9/127; A61K 9/51; A61K 31/7105; A61K 47/20; C12N 15/88

(52) U.S. Cl. CPC A61K 48/0041; A61K 9/0078; A61K 9/127; A61K 9/5123; A61K 31/7105; A61K 47/20; C12N 15/88

(57) ABSTRACT

The disclosure relates to compositions and methods for improving pulmonary delivery of therapeutic nucleic acids, e.g., DNA, mRNA, by enhancing transfection efficiency and thus efficacy of the therapeutic nucleic acids, using inhaled, Intranasal, Intratracheal, bronchial instillation and/or topical administration Intratracheal, bronchial instillation and/or topical administration transfection efficiency enhancing agents, including but not limited to 2-mercaptoethane sulfonate or cysteamine.

### CROSS REFERENCE TO RELATED APPLICATIONS

This application claims priority of Provisional Application No. 63/547,629 filed on Nov. 7, 2023, and entitled “METHODS AND FORMULATIONS FOR DELIVERY OF NUCELIC ACIDS” the entirety of which is incorporated herein by reference.

### FIELD OF THE INVENTION

The present invention generally relates to the field of therapeutic and prophylactic nucleic acid delivery to cells within the respiratory system. In particular, the present invention is directed to Agents, compositions and methods for enhanced delivery of nucleic acids to lungs and airway cells.

Approximately 34 million people in the US and more than half a billion people worldwide live with pulmonary diseases. Some of those diseases do not have any available treatment, such as Primary Ciliary Dyskinesia (PCD), Idiopathic Pulmonary Fibrosis, and certain lung cancers.

### BACKGROUND

The methods, agents, compositions, and technology platforms to improve the transfection efficiency of Nucleic Acids (Ribonucleic Acid, RNA, or Deoxyribonucleic acid, DNA) into cells of the respiratory system have the potential to unleash the limitless potential of nucleic acid vaccines and therapeutics for the effective and targeted prevention and treatment of lung diseases. This would be applicable to nucleic acid, including RNA and DNA-based vaccines and therapies delivered via various delivery vehicles to reach into the target cells and exert their effects, including Lipid Nanoparticles (LNP), Viral Vectors, Polymeric Nanoparticles, Exosomes or extracellular vesicles, Peptide-based Vehicles, Gold Nanoparticles, Cell-penetrating Peptides and Dendrimers or by using electroporation.

### SUMMARY OF THE DISCLOSURE

In an aspect, a composition for enhanced delivery of nucleic acids to tissues and cells of respiratory system for preventing and treating pulmonary disease is comprised of at least a transfection efficiency enhancing agent and a therapeutic nucleic acid in or attached to a delivery vehicle.

In an aspect, a method of enhanced delivery of nucleic acids to cells within the respiratory system for the treatment of pulmonary diseases, the method comprising administering a formulation comprising of a transfection efficiency enhancing agent and a therapeutic nucleic acid in or attached to a delivery vehicle to a subject in need thereof and wherein the transfection efficiency enhancing agent enhances the delivery of the therapeutic nucleic acid to cells within the respiratory system.

In an aspect, a method of enhanced delivery of nucleic acids to cells within the respiratory system, the method comprising administering a first formulation comprising a transfection efficiency enhancing agent to a subject in need thereof and administering a second formulation comprising a therapeutic nucleic acid in or attached to a delivery vehicle to the subject in need thereof.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the following drawings show aspects of one or more embodiments of the invention. However, it should be understood that the present invention is not limited to the precise arrangements and instrumentalities shown in the drawings, wherein:

FIG. 1 is a diagram of an exemplary embodiment of compositions for enhanced delivery of nucleic acids to cells within the respiratory system.

FIG. 2 is a flowchart of an exemplary embodiment of a method for preventing and/or treating pulmonary diseases.

FIG. 3 is a flowchart of an exemplary embodiment of a method for preventing and/or treating pulmonary diseases.

FIG. 4 is an illustration of an exemplary embodiment of cellular toxicity/safety data of transfection-enhancing agents in primary human airway cells;

FIG. 5 is an illustration of an exemplary embodiment of cellular toxicity data in various cell lines;

FIG. 6 is an illustration of an exemplary embodiment of the percentage increase in transfection of nucleic acid in the cells within the respiratory system with and without prior exposure to transfection-enhancing agent; and

FIGS. 7A-7C are an illustration of an exemplary embodiment of various methods, compositions and administration sequences for improving transfection efficiency and delivery of nucleic acid to the tissues and cells of the respiratory systems

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

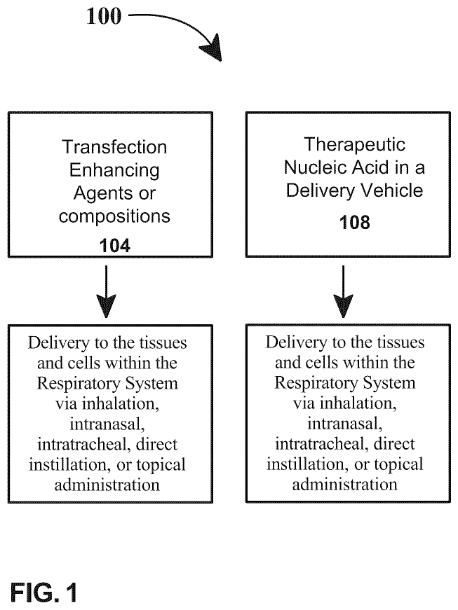

Referring now to FIG. 1, an exemplary embodiment of a composition for enhanced delivery of nucleic acids to lungs and airway cells, 100 is illustrated. Composition 100 includes A “transfection efficiency enhancing agent 104,” as used in this disclosure, is any compound or composition that aids in the delivery of therapeutic agents to tissues in the respiratory system. A “therapeutic agent,” as used in this disclosure, is any agent administered to the cells or tissues for treating or preventing a disease, with or without a delivery vehicle, in conjunction with a transfection efficiency enhancing agent 104. Therapeutic agents may include a second transfection efficiency enhancing agent 104. Therapeutic agent may include a therapeutic nucleic acid 108 as described below in more detail. The therapeutic agent may be prepared in combination with transfection efficiency enhancing agent 104 and, in a single formulation and administration, may be administered concurrently with transfection efficiency enhancing agent 104 in separate formulations, at a time point after administration of transfection efficiency enhancing agent 104, at the same time as administration as transfection efficiency enhancing agent 104, before administration of transfection efficiency enhancing agent 104, and the like as described below in more detail. Transfection enhancing agents may include amino acid derivatives, peptides, peptide analogues, and/or other small molecules as active agents, alone or in combination with one or more of the agents. Transfection efficiency enhancing agent 104 may be administered as co-therapy or a fixed-dose combination with a therapeutic nucleic acid 108. Transfection efficiency enhancing agent 104 may be delivered to the respiratory system via inhalation, intranasal, intratracheal, and/or direct instillation. “Cells within the respiratory system” as used in this disclosure are any cells that make up any cells in the tissues and organs in the upper and lower respiratory system in the human body, including the nose, mouth, sinuses, pharynx, and larynx trachea, bronchi, alveoli and lungs. The “respiratory system” as used in this disclosure includes any collection of organs that allow breathing such as the upper respiratory tract including the nose, mouth, sinuses, pharynx, and larynx and the lower respiratory tract including the trachea, bronchi, alveoli and lungs. Cells within the respiratory system may include but are not limited to epithelial cells, alveolar cells, endothelial cells, ciliated cells, goblet cells, macrophages, basal cells, club cells, brush cells, smooth muscle cells, fibroblast, dendritic cells, mast cells, macrophage, neutrophils, eosinophils and/or neuroendocrine cells. In an embodiment, cells within the respiratory system may include any cell located within the respiratory systems of a human body.

With continued reference to FIG. 1, transfection efficiency enhancing agent 104 may be selected from all forms, formulations, and compositions of the list of following compounds:
2-Mercaptoethane Sulfonate (MESNA) in concentration from about 5% to 95%, such as 5%, 10%, 15%, 20%, 95%, or any concentration in between, as the invention is not limited in this regard;
2-Aminoethane-1-thiol (Cysteamine) in concentration from about 5% to 95% such as 5%, 10%, 10%, 15%, 20%, 95%, or any concentration in between as the invention is not limited in this regard;
L-α-ureido-mercaptopropionic acid in concentrations from about 5 mg/ml to about 250 mg/ml, such as 20 mg/ml, 100 mg/ml, 150 mg/ml, 200 mg/ml, 250 mg/ml or any other concentration within this range as the invention is not limited in this regard;
Bromhexine;
Ascorbic acid, such as Vitamin-C (reduced Ascorbic acid or an ascorbate salt thereof);
N-Acetylcysteine;
N-Butylcysteine;
Reduced glutathione, such as natural tri-peptide glutathione in the reduced form of glutathione;
N-derivatives and C-derivates of amino acid cysteine;
Di-peptide of cysteine and glutamic acid;
Di-peptide of aspartic acid;
Ambroxol hydrochloride;
Vitamin E
Tris(2-carboxyethyl) phosphine hydrochloride
DNase; such as recombinant DNase;
DNA cleaving agent

With continued reference to FIG. 1, in particular embodiments, transfection

efficiency enhancing agent 104 may include a thiol moiety, for example, sodium 2-mercapto ethane sulfonate (MESNA) or 2-Aminoethane-1-thiol/β-Mercaptoethylamine, (Cysteamine), administered in therapeutically active reduced (free thiol-SH) form.

In particular embodiments, transfection efficiency enhancing agent 104 and compositions thereof may include a sulfonate moiety (SO₃) or any other charged functional group, for example, sulfonate moiety (SO₃) in sodium 2-mercaptoethane sulfonate (MESNA) or for example an amine moiety (NH₂) in 2-Aminoethane-1-thiol (Cysteamine), in addition to thiol moiety in active reduced (free thiol —SH) form.

In some embodiments, a thiol moiety may break disulfide bonds within the highly viscoelastic mucus barrier or biofilm, in patients suffering from various lung diseases. The second functional group, in the case of MESNA, the sulfonate group may act in synergy with the thiol group in dispersing the mucus barrier by also disrupting hydrogen bonds, Vander-wall interaction and other electrostatic interactions. The amine group in the case of Cysteamine may provide a similar function.

With continued reference to FIG. 1, a transfection efficiency enhancing agent 104 or composition thereof may be in any form including but not limited to solution form, gels, drops or aerosolized sprays for inhaled, intranasal, intratracheal, bronchial instillation and/or topical administration and/or in dry-powder microspheres form and encapsulated in lipids as microparticle or nanoparticles for DPI delivery with dry powder inhalers including, single dose inhaler, multi-dose inhalers, metered-dose inhalers (MDIs), Breath-Actuated inhaler, Digital and Smart dry powder inhalers, inhalers with or without Hydrofluoroalkanes (HFAs) and/or compressed gases for the pressurized spray to create a fine aerosol.

In some embodiments, a transfection efficiency enhancing agent 104 or composition thereof may be delivered via a nebulizer, spay or dry powder inhaler, metered dose inhaler, breath-actuated inhaler, digital/smart dry powder inhalers, nasal spray, droppers, or any other suitable device.

In some embodiments, delivery methods and/or delivery vehicles, formulating compounds in the transfection efficiency enhancing agent described herein may aid in maintaining a compound in its therapeutically active form.

In some embodiments, a suitable dose of transfection-enhancing agent 104 and formulation for delivery via Inhalation, intranasal, intratracheal, bronchial instillation and/or topical administration route may be in the range of 0.15-150 mg for co-administration or pre-administration with therapeutic nucleic acids with or without a delivery vehicle.

In some embodiments, a suitable dose of a transfection enhancing agent 104 or composition or formulation thereof may be in the range of 0.5-60 mg, for delivery via inhalation, intranasal, intratracheal, bronchial instillation and/or topical administration route.

In some embodiments, a transfection efficiency enhancing agent 104 or composition thereof may be administered at the same time as a therapeutic nucleic acid 108.

In some embodiments, transfection efficiency enhancing agent 104 or composition may be administered prior to administration of a therapeutic nucleic acid 108, such as 5-60 minutes prior to administration of a therapeutic nucleic acid 108.

In some embodiments, a transfection efficiency enhancing agent 104 or composition thereof may be administered in a single dose combination with a therapeutic nucleic acid 108 in a vehicle. For example, transfection efficiency enhancing agent 104 or composition in microspheres/nanospheres may be combined with nanoparticles or nanospheres of therapeutic nucleic acid in or attached to the delivery vehicle.

Inhalable particles comprising transfection efficiency enhancing agent 104 or their compositions may be manufactured via lipid encapsulation by multiple methods including micronization, Ball Milling, Air-jet Milling, crystallization, Electrohydrodynamic Atomization, Coacervation, Fluidized Bed Granulation, Emulsion-Solvent Evaporation, Supercritical Fluid Technology, spray-drying, spray-freezing, a solution of active drug substance formulated with any lipid including but not limited to phospholipid (e.g. phosphatidylcholine, such as 1,2-distearoyl-sn-glycero-3-phosphocholine (DSPC) or dipalmitoyl phosphatidylcholine (DPPC), Dioleoylphosphatidylethanolamine (DOPE), and Dipalmitoylphosphatidylglycerol (DPPG)), Cationic lipids (e.g. 1,2-Dioleoyl-3-trimethylammonium-propane (DOTAP), Dimethyldioctadecylammonium bromide (DDAB), Ionizable Lipids, DLin-MC3-DMA (MC3), DOBAQ, DLin-KC2-DMA, Neutral lipids: Dioleoylphosphatidylcholine (DOPC), Cholesterol, Phytosterols, Surfactants Lipids Lecithin, Phosphatidylserine (PS) Polyethylene glycol (PEG) lipids, Sphingolipids: Sphingomyelin, Ceramides, Fatty Acids and Triglycerides: Oleic acid, Stearic acid, Tristearin, or any other lipid and combination thereof.

With continued reference to FIG. 1, lipids contained within composition 100, may include but are not limited to one or more of the following lipids:

Phospholipids including for example:
1. Dipalmitoylphosphatidylcholine (DPPC): Primary component of lung surfactants and liposomes for inhalation; stabilizes bilayers in liposomal formulations.
2. Dioleoylphosphatidylcholine (DOPC): Enhances fluidity and is widely used in liposomes for drug delivery.
3. Dioleoylphosphatidylethanolamine (DOPE): Often combined with cationic lipids in lipid nanoparticles to improve cell membrane fusion, especially for gene and RNA delivery.
4. Dipalmitoylphosphatidylglycerol (DPPG): Used to stabilize liposomes and improve encapsulation efficiency, commonly for inhalation therapies.
5. Phosphatidylserine (PS): Helps mimic natural cell membranes and can be used to enhance uptake by specific cell types.

Cationic Lipids including for example:
1. 1,2-Diolcoyl-3-trimethylammonium-propane (DOTAP): Positively charged lipid used to complex with nucleic acids for efficient gene delivery.
2. Dimethyldioctadecylammonium Bromide (DDAB): Used in DNA and RNA delivery, forming stable complexes with negatively charged nucleic acids.
3. N,N-Dioleyl-N,N-dimethylammonium chloride (DODAC): A cationic lipid used for transfection and gene therapy applications.
4. DC-Cholesterol: Used in gene delivery formulations, especially in liposomal gene therapies, enhancing cellular uptake.

Ionizable Lipids including for example:
1. DLin-MC3-DMA (MC3): Gold standard in mRNA vaccines and LNPs for gene therapy due to its favorable ionization at acidic pH and reduced toxicity.
2. DOBAQ (1,2-Dioleoyloxy-3-(dimethylamino) propane): Used in lipid nanoparticles (LNPs) for mRNA and RNA interference (RNAi) therapies.
3. DLin-KC2-DMA: Common in LNP formulations, especially for siRNA delivery, due to its ability to efficiently escape the endosome.

Neutral Lipids including for example:
1. Dioleoylphosphatidylethanolamine (DOPE): A common helper lipid that enhances the fusion of liposomes with cell membranes.
2. Dioleoylphosphatidylcholine (DOPC): Used to stabilize liposomal membranes in gene therapy formulations.
3. Cholesterol: Increases membrane stability and reduces permeability in liposomes and lipid nanoparticles.
4. Squalene: Natural lipid used in some emulsions as a stabilizer and enhancer of drug delivery.

PEGylated Lipids (Polyethylene Glycol Conjugated Lipids) including for example:
1. DSPE-PEG2000 (1,2-Distearoyl-sn-glycero-3-phosphoethanolamine-N-[methoxy (polyethylene glycol)-2000]): Reduces immunogenicity, increasing circulation time and stability of lipid nanoparticles.
2. DMPE-PEG (1,2-Dimyristoyl-sn-glycero-3-phosphoethanolamine-N-[methoxy (polyethylene glycol)]): Often used in PEGylated liposomes for drug delivery and vaccine formulations.
3. C16-PEG Ceramide: Provides stability in lipid formulations and extends blood circulation times for liposomes and LNPs.

Sterols including for example:
1. Cholesterol: Adds rigidity and stability to lipid bilayers and is essential in the formation of liposomes and LNPs.
2. Ergosterol: Occasionally used in place of cholesterol in fungal formulations; stabilizes liposomal membranes.
3. Phytosterols: Natural sterols derived from plants, sometimes used as cholesterol substitutes in formulations.

Sphingolipids including for example:
1. Sphingomyelin: Adds stability to lipid nanoparticles and liposomal membranes, mimicking cell membrane composition.
2. Ceramides: Used in drug delivery formulations to enhance stability and retention of drugs within the lipid bilayer.

Fatty Acids and triglycerides including for example:
1. Oleic Acid: Helps stabilize formulations and improve liposome membrane fluidity.
2. Stearic Acid: Used in solid lipid nanoparticles (SLNs) for drug encapsulation and controlled release.
3. Tristearin: Commonly used in SLNs as a matrix-forming lipid for sustained drug release.
4. Palmitic Acid: Stabilizes SLNs and liposomal formulations for improved delivery.
5. Linoleic Acid: Occasionally used to increase membrane flexibility in lipid formulations.

Emulsifying and Surfactant Lipids including for example:
1. Lecithin: Commonly used in emulsions and liposomes as a natural emulsifier and stabilizer.
2. Monoolein: Used in the formation of lipid-based mesophases, such as cubosomes, for controlled drug delivery.
3. Sorbitan Monostearate: Stabilizes emulsions and provides a lipid source in topical and inhaled formulations.

Other Specialized Lipids including for example:
1. DOTMA (N-[1-(2,3-Dioleyloxy) propyl]-N,N,N-trimethylammonium chloride): Common in older cationic lipid formulations for gene delivery.
2. Archaea-derived lipids: Unusual ether lipids with high stability, occasionally explored in liposomes for drug delivery.
3. GM1 and GM3 Gangliosides: Glycolipids sometimes included to stabilize liposomes and target specific cell receptors.

In some embodiments, a transfection efficiency enhancing agent 104 or their composition may include a an additional compound including but not limited to calcium chloride (CaCl₂, NaCl, Aspartic acid or its salt and/or sugar such as lactose, maltodextrin, mannitol, trehalose, or mixtures thereof) to form low density particle for optimal aerodynamic and flight properties to easily reach the lower airway via multiple different kind of devices including nasal spray, droppers, nebulizers, dry powder inhalers including, single dose inhaler, multi-dose inhalers, metered-dose inhalers (MDIs), Digital and Smart dry powder inhalers, inhalers with or without Hydrofluoroalkanes (HFAs) and/or compressed gases for the pressurized spray to create a fine aerosol or Breath-Actuated inhalers that are effective even at low inspiratory flow rate that is common in patients of pulmonary diseases involving airway obstruction.

With continued reference to FIG. 1, in an embodiment, composition 100 may include a transfection enhancing agent such as sodium 2-mercaptoethane sulfonate (MESNA), or Cysteamine.

In some embodiments, transfection efficiency enhancing agent 104 or compositions, e.g., MESNA or Cysteamine, may be used in a method of enhancing transfection of nucleic acid in tissues and cells of the respiratory system.

In some embodiments, a solution of composition 100 including (MESNA or Cysteamine may be formulated for inhaled, intranasal, intratracheal, bronchial instillation or topical administration for delivery via suitable device.

In some embodiments, of composition 100, MESNA or Cysteamine may be formulated for Intranasal delivery in solutions, liquids, gels (for delivery via nasal sprays, droppers), topical delivery in Gels, Ointment, Transdermal patches, inhaled delivery in solutions (via nebulizer, spray), in dry-powders (for delivery via DPI inhalers or pressurized inhalation devices) and intratracheal or direct instillation (via a syringe or sprays).

With continued reference to FIG. 1, in another embodiment, transfection efficiency enhancing agent 104 or compositions may include a spray-dried microsphere formulation comprising sodium 2-mercaptoethane sulfonate (MESNA) or 2-mercapto ethylamine (Cysteamine), for example a spray-dried microsphere formulation comprising 5%-90% MESNA or Cysteamine by weight of the formulation, e.g., comprising about 85% MESNA or Cysteamine by weight of the formulation.

With continued reference to FIG. 1, disclosed herein are inhalable particles of a lipid-encapsulated transfection efficiency enhancing agents or composition (e.g., dry-powder microspheres of 2-mercaptoethanol sulfonate (MESNA) or 2 mercapto ethylamine (Cysteamine) which may improve the delivery and/or transfection efficiency of therapeutic nucleic acid 108 formulated with or without delivery vehicle (e.g., DNA, Plasmid DNA, RNA, including messenger RNA (mRNA), small interfering RNAs (siRNA), antisense oligonucleotides (ASO), aptamers, micro RNA (miRNA), transfer RNA (tRNA), Self-Amplifying RNA (saRNA), Gene Editing Components (e.g., CRISPR-Cas9), gene editing mRNA components, Guide RNA (gRNA) for CRISPR Systems, prime editing mRNA components, or circular RNA.

With continued reference to FIG. 1, in some embodiments, transfection efficiency enhancing agent 104 or compositions may include a spray-dried microsphere formulation comprising MESNA or Cysteamine, DSPC, and CaCl₂. For example, a spray-dried microsphere may comprise (i) 15-90% w/w MESNA or Cysteamine; 10-50% w/w DSPC; and 3-10% CaCl₂; e.g., 49.5% w/w MESNA or Cysteamine; 47.3% w/w DSPC; and 3.2% CaCl₂ or (ii) 30-35% w/w MESNA or Cysteamine, 60-65% w/w DSPC, and 3-5% w/w CaCl₂; e.g., 32.95% w/w MESNA or Cysteamine, 62.8% w/w DSPC, and 4.2% w/w CaCl₂. Particles may be optimized, e.g., by using a microfluidizer. This may be done to obtain a uniform population of microparticles (such as a uniform microparticle size and/or optimal Mass Median Acrodynamic Diameter, MMAD). In some embodiments, microparticles may have a mass median aerodynamic diameter between 100 nm or 0.1 μm and 10 μm, e.g. 100 nm or 0.1 μm to 7 μm, 3 μm to 5 μm, and so on. In some embodiments, a dose may be in the range of 10 μg to 150 mg. Total dose amounts may be delivered as a single dose or spread over multiple doses with defined intervals.

With continued reference to FIG. 1, transfection efficiency enhancing agent 104 and composition may include a delivery vehicle such a liposome, nanoparticle or microsphere formulation. A “liposomal formulation” as used in this disclosure, is a delivery system in which any agent for example a transfection efficiency enhancing agent 104 is encapsulated within a liposome. A “liposome” as used in this disclosure includes a vesicle composed of one or more lipids as bilayers that encapsulate an aqueous core and is used to protect agent or drug products from degradation or immune system attack and facilitate targeted delivery, biocompatibility, and the like. The liposomal formulation may be uniquely tailored for and administered directly to the target location in the respiratory tract via Inhaled, Intranasal, Intratracheal, bronchial instillation or topical delivery to the site of pathology, thereby avoiding systemic drug exposure caused when drugs or agents are administered intravenously, intraarterially, intramuscularly, or in oral formulation. This may allow for a reduced or smaller dose of these agents, which may alleviate many side effects associated therewith. With continued reference to FIG. 1, A “microsphere” formulation as used in this disclosure, is a delivery vehicle in 1-1000 μm size, in which any agent for example a transfection efficiency enhancing agent 104 is encapsulated in a microsphere with solid or porous core and designed for controlled or sustained release of drugs and offer structural stability and controlled degradation.

With continued reference to FIG. 1, liposomes, microsphere or lipid nanoparticles may represent unique carriers/vehicles that can site-specifically deliver the drug while protecting it from interaction with the environments (blood, metabolism, exposure to air, etc.) or modification. As such these vehicles can also protect transfection efficiency enhancing agent 104 or composition from pre-delivery interaction or modifications, thereby preserving its potency until the drug is released at the target site. When delivered to the target site, lipids and other components in the formulation may further support transfection by disrupting hydrophilic and/or hydrophobic interactions within and between various biological components including proteins as well as penetrating mucus layer.

With continued reference to FIG. 1, mucus may serve as a form of protection of respiratory cells or envelope the cells in the respiratory tract, whereby a therapeutic nucleic acid 108 may be unable to reach inside the cells within the respiratory system and exert its effect. Disrupting the structured mucus and its subsequent liquefication may enable a therapeutic nucleic acid 108 to reach the cytoplasm of lungs and airway cells and exert its effects.

With continued reference to FIG. 1, a “therapeutic nucleic acid 108” as used in this disclosure, is a nucleic acid DNA or RNA in or attached to a delivery vehicle designed to prevent, treat or modify a disease process. A therapeutic nucleic acid includes any RNA or DNA within or attached to a delivery vehicle as cargo. DNA or RNA including but are not limited to Plasmid DNA, RNA, including messenger RNA (mRNA), small interfering RNAs (siRNA), antisense oligonucleotides (ASO), aptamers, micro-RNA (miRNA), transfer RNA (tRNA), Self-Amplifying RNA (saRNA), Gene Editing Components (e.g., CRISPR-Cas9), gene editing mRNA components, Guide RNA (gRNA) for CRISPR Systems, prime editing mRNA components, and circular RNA

With continued reference to FIG. 1, a therapeutic nucleic acid 108 may be used to modulate gene expression, repair genetic mutations, regulate protein production to achieve desired preventive and therapeutic effects, and the like. The administration of therapeutic nucleic acid 108s may be used to, for example, supplement essential proteins or edit genes could potentially be very effective in addressing lung pulmonary diseases as it offers a high concentration in the lungs and permits minimal systemic exposure and related adverse effects. This may be done, for example, in cells within the respiratory system.

With continued reference to FIG. 1, in some embodiments, a therapeutic nucleic acid 108 may modulate expressions of cytokines that cause inflammation, e.g., IL-4, IFN-β, or IL-10; e.g., Xalud's, XT-150, a DNA plasmid that expresses a modified form of IL-10, e.g., administered to a patient suffering from an inflammatory disease, e.g., asthma or COPD. In some embodiments, a therapeutic nucleic acid 108 may encode a tumor suppressor gene product, e.g., p53 or tumor suppressor candidate 2 (TUSC2), e.g., administered to a patient suffering from lung cancer, e.g., non-small cell lung cancer.

A therapeutic nucleic acid 108, RNA and DNA, may be used as a vaccine and/or therapeutic for the prevention or treatment of a disease. A therapeutic nucleic acid 108 may be delivered to the respiratory system via inhalation, intranasal, intratracheal, direct instillation, and/or topical administration. A “delivery vehicle,” as used in this disclosure, is any tool, formulation, structure, methodology, substance, and/or composition used to introduce therapeutic nucleic acid 108 into cells within the respiratory system. Delivery vehicles may include but are not limited to viral vectors, lipid nanoparticles, liposomes, polymeric nanoparticles, exosomes, extracellular vesicles, peptide-based vehicles, gold nanoparticles, cell penetrating peptides and dendrimers or by using electroporation, and the like.

With continued reference to FIG. 1, a therapeutic nucleic acid 108 may include DNA and/or RNA. In non-limiting examples, a therapeutic nucleic acid 108 may include DNA, Plasmid DNA, messenger RNA (mRNA), small interfering RNAs (siRNA), antisense oligonucleotides (ASO), aptamers, micro RNA (miRNA), transfer RNA (tRNA), Self-Amplifying RNA (saRNA), Gene Editing Components (e.g., CRISPR-Cas9), gene editing mRNA components, Guide RNA (gRNA) for CRISPR Systems, prime editing mRNA components, or circular RNA). RNA therapies may offer particular advantages, such as low toxicity, high specificity and low risk of off-target mutagenesis. However, RNA may have limitations, such as potential degradation by RNases, and free RNA may provoke a strong immune response.

Therapeutic nucleic acid 108 may include a prophylactic nucleic acid. A “prophylactic nucleic acid,” as used in this disclosure, is any therapeutic nucleic acid 108 used for the prevention of any pulmonary and/or respiratory disease or condition, including any disease or condition described below in reference to drawings FIGS. 1-7 herein. A prophylactic nucleic acid may include, for example, a vaccine. For instance, and without limitation, a prophylactic nucleic acid may include COMIRNATY (COVID-19 Vaccine, mRNA) as produced by Pfizer-BioNTech of NEW YORK, UNITED STATES, and/or SPIKEVAX (COVID-1A9 Vaccine, mRNA) as produced by Moderna of MASSACHUSETTS, UNITED STATES. Additional examples, for instance, and without limitation, a therapeutic nucleic acid may include, for example, an mRNA influenza vaccine, RSV vaccine, Zika vaccine, HIV vaccine, and vaccine for other infectious diseases.

Therapeutic nucleic acid composition 108 may include a therapeutic nucleic acid. A “therapeutic nucleic acid,” as used in this disclosure, is any therapeutic nuclei-[acid used for treating any pulmonary and/or respiratory disease or condition, including any disease or condition described below in reference to the drawings FIGS. 1-7 here in. For instance, and without limitation, a therapeutic nucleic acid used for treatment may include mRNA, siRNA, or ASO for example, therapeutic nucleic acid treatments for primary Ciliary Dyskinesia are being developed by ReCode OF CALIFORNIA, UNITED STATES, TranslateBio/SANOFI OF MASSACHUSETTS, UNITED STATES, Ethris GMBH OF GERMANY or for example-an antisense oligonucleotide approved for the treatment of cytomegalovirus (CMV) retinitis in AIDS patients.

In some embodiments, A therapeutic agent, for example, RNA, may be modified to improve stability and effectiveness and/or to reduce immunogenicity, for example, by using lipid nanoparticles as a delivery agent or by including atypical nucleotides (e.g., pseudo uridine in place of uridine, 5-methylcytidine in place of cytidine, 1-methylpseudouridine in place of uridine, or other atypical/unusual nucleotides in addition to (or instead of) cytidine, uridine, adenosine, and guanosine). Inclusion of atypical nucleotides may be done as described in WO2007024708A2, which is incorporated herein by reference in its entirety. In some embodiments, polynucleotides may be modified by altering terminal regions of RNA, e.g., a 5′ cap and/or a 3′ poly-A tail, circularization, and/or by other modifications as known in the art.

With continued reference to FIG. 1, in some embodiments, a therapeutic nucleic acid 108 may be encapsulated with a lipid as a spray-dried nanosphere or microsphere, e.g., a microsphere/nanosphere including mRNA, a lipid (e.g., DSPC), and/or another substance (e.g., CaCl2).

In some embodiments, a therapeutic nucleic acid 108 may be provided in or attached to a vehicle including but not limited to Lipid Nanoparticles (LNP), Viral Vectors, Polymeric Nanoparticles, Exosomes or extracellular vesicles, Peptide-based Vehicles, Gold Nanoparticles, Cell-penetrating Peptides Dendrimers and or by using electroporation, and the like.

In some embodiments, lipid nanoparticles may include but are not limited to an ionizable lipid, a non-cationic lipid, a cationic lipid, a sterol, and a PEGylated lipid. In some embodiments, an ionizable lipid is selected from ALC-0315, SM-102, and MC3. In some embodiments, a phospholipid may include 1,2-distearoyl-sn-glycero-3-phosphocholine (a.k.a. distearoylphosphatidylcholine or DSPC). In some embodiments, a PEGylated lipid may be selected from DMG-PEG1000-15,000. In some embodiments, a therapeutic nucleic acid 108 may be delivered via a viral vector, e.g., an adenovirus or adeno-associated virus, as a vehicle.

In some embodiments, a therapeutic nucleic acid 108 may include mRNA modified to improve stability of RNA. In some embodiments, a therapeutic nucleic acid 108 may include mRNA including a 5′ cap and/or a 3′ poly-A tail.

With continued reference to FIG. 1, in certain embodiments, therapeutic nucleic acids and transfection efficiency enhancing agents may each be formulated in spray-dried nanospheres/microspheres, which may be combined for co-administered or administered in sequence. For example, an inhalable composition may include (i) spray-dried microspheres, nanospheres/nanoparticles including a nucleic acid, comprising mRNA, a lipid including but not limited to a phospholipid, for example a phosphatidylcholine, such as 1,2-distearoyl-sn-glycero-3-phosphocholine (DSPC) or dipalmitoyl phosphatidylcholine (DPPC), Dioleoylphosphatidylethanolamine (DOPE), and Dipalmitoylphosphatidylglycerol (DPPG)), Cationic lipids such as 1,2-Dioleoyl-3-trimethylammonium-propane (DOTAP), Dimethyldioctadecylammonium bromide (DDAB), Ionizable Lipids: DLin-MC3-DMA (MC3), DOBAQ, DLin-KC2-DMA, Neutral lipids: Dioleoylphosphatidylcholine (DOPC), Cholesterol, Phytosterols Surfactants Lipids: Lecithin, Phosphatidylserine (PS) Polyethylene glycol (PEG) lipids, Sphingolipids: Sphingomyelin, Ceramides, Fatty Acids and Triglycerides: Oleic acid, Stearic acid, Tristearin, or any other lipid and their combination suitable for similar purposes.

With continued reference to FIG. 1, in some embodiments, transfection efficiency enhancing agent 104 or compositions may include a thiol in reduced form and/or active form.

With continued reference to FIG. 1, in certain embodiments, therapeutic nucleic acids and transfection efficiency enhancing agents may each or both together be formulated with additional agents including but not limited to Water, CaCl2, Ethanol, Glycerin, NaCl, Lactose, Mannitol, Propellants; Hydrofluoroalkanes (HFAs) Compressed Gases, Surfactants, Emulsifiers; Polysorbates Sorbitan esters, Lecithin, pH adjusters, Viscosity Modifiers, Carboxymethylcellulose (CMC), Hyaluronic Acid, Polyvinyl Alcohol, Preservatives and Antimicrobial Agents; Benzalkonium Chloride, Phenoxyethanol and Parabens, EDTA (Ethylenediaminetetraacetic Acid, Stabilizer Ascorbic Acid (Vitamin C), Sodium Metabisulfite, Tocopherol (Vitamin E).

In some embodiments, transfection efficiency enhancing agent 104 or compositions may include a spray dried microsphere of sodium 2-mercaptoethane sulfonate. In some embodiments, transfection efficiency enhancing agent 104 or compositions may include a spray dried microsphere of Cysteamine. In some embodiments, transfection efficiency enhancing agent 104 or compositions may include DNase, e.g., a spray dried microsphere of DNase.

With continued reference to FIG. 1, in some embodiments, a therapeutic nucleic acid 108 may be in or attached to a viral vector, e.g., an adenovirus or adeno-associated virus.

In some embodiments, a therapeutic nucleic acid 108 may include RNA, e.g., selected from mRNA and siRNA including messenger RNA (mRNA), mRNA vaccines, small interfering RNAs (siRNA), antisense oligonucleotides (ASO), aptamers, micro RNA (miRNA), transfer RNA (tRNA), Self-Amplifying RNA (saRNA), Gene Editing Components (e.g., CRISPR-Cas9), gene editing mRNA components, Guide RNA (gRNA) for CRISPR Systems, prime editing mRNA components, and circular RNA and may be administered in a lipid nanoparticle. In some embodiments, a lipid nanoparticle may include lipids including but not limited to ionizable lipids (e.g., a tertiary amine), Cationic lipids, non-cationic lipids (e.g., phospholipid and cholesterol), and PEGylated lipids. In some embodiments, an ionizable lipid may be selected from e.g. DLin-MC3-DMA DOBAQ, DLin-KC2-DMA, ALC-0315, SM-102, and MC3. In some embodiments, a phospholipid may include 1,2-distearoyl-sn-glycero-3-phosphocholine (a.k.a. distearoylphosphatidylcholine or DSPC). In some embodiments, a PEGylated lipid may be selected from DMG-PEG1000, 15000 and ALC-0159.

With continued reference to FIG. 1, composition 100 may include a liposomal formulation, microsphere, nanoparticles, and/or engineered respirable particles of therapeutic agents.

With continued reference to FIG. 1, in some embodiments, a therapeutic nucleic acid 108 may include RNA, e.g., selected from mRNA and siRNA including messenger RNA (mRNA), mRNA vaccines, small interfering RNAs (siRNA), antisense oligonucleotides (ASO), aptamers, micro RNA (miRNA), transfer RNA (tRNA), Self-Amplifying RNA (saRNA), Gene Editing Components (e.g., CRISPR-Cas9), gene editing mRNA components, Guide RNA (gRNA) for CRISPR Systems, prime editing mRNA components, and circular RNA In some embodiments, a therapeutic nucleic acid 108 may include mRNA modified to contain pseudouridine in place of uridine, 5-methylcytidine in place of cytidine, and/or 1-methylpseudouridine in place of uridine. In some embodiments, a therapeutic nucleic acid 108 may include mRNA including a 5′ cap and/or a 3′ poly-A tail.

In some embodiments, therapeutic nucleic acid 108 may include mRNA coding a normal/healthy version of the protein, which may be administered to treat a patient suffering from a pulmonary disease due to a defective gene that can cause any lung disease, e.g., cystic fibrosis caused by a defective CFTR gene or primary ciliary dyskinesia caused by a defective DNAH5 gene.

With continued reference to FIG. 1, in some embodiments, a therapeutic nucleic acid 108 may include RNA, e.g., selected from mRNA and siRNA including but not limited to antisense oligonucleotides (ASO), aptamers, micro RNA (miRNA), transfer RNA (tRNA), Self-Amplifying RNA (saRNA), Gene Editing Components (e.g., CRISPR-Cas9), gene editing mRNA components, Guide RNA (gRNA) for CRISPR Systems, prime editing mRNA components, or circular RNA).

In some embodiments, a therapeutic nucleic acid 108 may include DNA or mRNA modified to contain pseudo uridine in place of uridine, 5-methylcytidine in place of cytidine, and/or 1-methylpseudouridine in place of uridine. In some embodiments, a therapeutic nucleic acid 108 may include but not limited to mRNA encoding normal healthy CFTR gene or DNAI1.CCDC39, CCDC40 or any other gene involved in causing any genetic pulmonary disease.

With continued reference to FIG. 1, in some embodiments, a spray-dried microsphere composition may include a therapeutic nucleic acid 108 (e.g., mRNA), transfection efficiency enhancing agent 104 or compositions (e.g., MESNA or Cysteamine), a lipid (e.g., a phospholipid, e.g., DSPC or DPPC), and calcium chloride). In some embodiments, composition of 100 in a spray-dried microsphere may include mRNA, MESNA or cysteamine, DSPC, and CaCl2. In some embodiments, a spray-dried nanosphere+microsphere composition may include mRNA with delivery vehicle (e.g. lipid nanoparticles), and MESNA, DSPC, and CaCl2. In some embodiments, a spray-dried microsphere composition may include mRNA, Cysteamine, DSPC, and CaCl2. In some embodiments, a spray-dried composition may include mRNA with a delivery vehicle (e.g., lipid nanoparticles/nanospheres and microspheres of cysteamine, DSPC, and CaCl2.

With continued reference to FIG. 1, in some embodiments, a composition for improved delivery and enhanced transfection of nucleic acids to cells within the respiratory system may include an inhalable formulation including (i) spray-dried composition including a nucleic acid, e.g., a spray-dried form including mRNA, formulated with a lipid (e.g., DSPC), and another agent (e.g., CaCl2); and (ii) spray-dried microspheres including transfection efficiency enhancing agent 104 or its compositions, e.g., a spray-dried microsphere including MESNA, a lipid (e.g., DSPC), and a carrier (e.g., CaCl2), or a spray-dried microsphere including Cysteamine, a lipid (e.g., DSPC), and a carrier (e.g., CaCl2).

With continued reference to FIG. 1, a composition or method may be consistent with those disclosed in international patent application WO2022047047A1, the entirety of which is incorporated herein by reference.

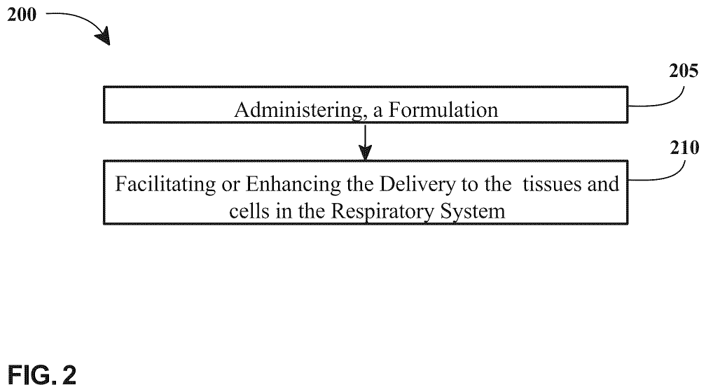

Referring now to FIG. 2, an exemplary embodiment of method 200 of enhanced delivery of nucleic acids to cells within the respiratory system for the treatment of pulmonary diseases is illustrated. At step 205, administration of a formulation comprising a transfection efficiency enhancing agent 104 and a therapeutic nucleic acid 108 are delivered via Inhalation or Intranasal, Intratracheal, bronchial instillation and/or topical administration to a subject in need thereof is illustrated. Transfection efficiency enhancing agent 104 includes any transfection efficiency enhancing agent 104 as described above in more detail in reference to FIG. 1. Therapeutic nucleic acid 108 includes any therapeutic nucleic acid 108 as described above in more detail in reference to FIG. 1. Administering a formulation may include administering transfection enhancing agent 104 and therapeutic nucleic acid 108 in or attached to a delivery vehicle via inhalation, intranasal, intratracheal, bronchial instillation and direct instillation or topical administration. In an embodiment, transfection-enhancing agent 104 and therapeutic nucleic acid 108 in or attached to the delivery vehicle may be administered simultaneously.

With continued reference to FIG. 2, in some embodiments, a therapeutic nucleic acid 108 may modulate expressions a cytokine that cause inflammation, e.g., IL-4, IFN-β, or IL-10; e.g., Xalud's, XT-150, a DNA plasmid that expresses a modified form of IL-10, e.g., administered to a patient suffering from an inflammatory disease, e.g., asthma or COPD. In some embodiments, a therapeutic nucleic acid 108 may encode a tumor suppressor gene product, e.g., p53 or tumor suppressor candidate 2 (TUSC2), e.g., administered to a patient suffering from lung cancer, e.g., non-small cell lung cancer.

With continued reference to FIG. 2, at step 210, transfection efficiency enhancing agent 104 enhances the delivery of therapeutic nucleic acid 108 to the tissues and cells within the respiratory system, as described above in more detail in reference to FIG. 1. Transfection efficiency enhancing agent 104 may be administered as co-therapy or a fixed-dose combination with a therapeutic nucleic acid 108. This may be performed utilizing any methodology or device as described above in more detail in reference to FIG. 1.

With continued reference to FIG. 2, “delivery” as used in this disclosure is any delivery mechanism whereby formulation comprising a transfection efficiency enhancing agent 104 and a therapeutic nucleic acid 108 are administered to the respiratory system. Delivery may include but is not limited to solutions, drops, gels, dry powder, gases, aerosols, fine powders, pulmospheres, microspheres, nanosphere, liposomes or lipid nanoparticles for direct administration to the respiratory system. Inhalation, intranasal intra-tracheal, intratracheal, bronchial instillation, and/or topical administration may include but is not limited to the use of a delivery device including but not limited to dry-powder inhalers, nebulizers, Nasal sprays, Droppers, metered dose inhalers. Digital smart inhalers, single dose inhalers, multidose inhalers, reservoir inhalers, masks and/or any other equipment, delivery mechanism, and/or any other device described herein. Inhalation of dry-powder form may include but is not limited to the use of an inhaler such as but not limited to a dry powder inhaler (DPI), a metered dose inhaler, a compressed gas inhaler, and/or any other suitable device.

Delivery via inhalation or intranasal administration of therapeutic nucleic acid 108, can be particularly challenging in some disease conditions such as Chronic Obstructive Pulmonary Diseases (COPD), severe asthma, primary ciliary dyskinesia, cystic fibrosis, and bronchiectasis as the mucus may be highly viscous, and clastic and pose a higher barrier to the delivery of therapeutic nucleic acid. Composition 100 containing a transfection efficiency enhancing agent in combination with a therapeutic nucleic acid such as RNA and DNA in or attached to a delivery vehicle such as lipid nanoparticles (LNP), viral vectors, polymeric nanoparticles, exosomes or extracellular vesicles, peptide-based vehicles, gold nanoparticles, cell-penetrating peptides, and dendrimers may provide a synergistic effect to break through mucus barriers and cell membrane to enter the cell and exert their effect on cells that may be contained within the Respiratory system and throughout the human body as described above in more detail in reference to FIG. 1.

With continued reference to FIG. 2, formulation comprising a transfection efficiency enhancing agent 104 and a therapeutic nucleic acid 108 may be delivered directly to the respiratory system via inhalation, intranasal, intratracheal, bronchial instillation, and/or topical route. The particles of the invention may be delivered by means of administering inhalable liposomes, microspheres, nanoparticles, engineered respirable particles, and aerosols directly to the respiratory system. As used in this disclosure, “inhalation” includes any delivery mechanism to tissues and cells within the respiratory system, including via the mouth or nose, such as oral inhalation and/or intranasal delivery and/or delivery via nasal passages to the upper and lower respiratory system.

The methods for this direct delivery via inhalation, Intranasal, Intratracheal, bronchial instillation and/or topical administration may include the use of a dispersing apparatus such as an Dry powder inhaler, metered dose inhaler, a handheld portable nebulizer or compressor-nebulizer, nasal sprayer device capable of delivering the drug particles via the inhaled and/or intranasal route. Furthermore, the particles of the transfection efficiency enhancing agent 104 and/or therapeutic nucleic acid 108 of the present invention may be stored in a dry powder form (such as a dry powder inhaler metered dose inhaler), a gel or ointment form or in a liquid/solution form (such as in vials for nebulizer).

In some embodiments, transfection efficiency enhancing agent 104 or compositions, e.g., MESNA or Cysteamine, may be used in a method of enhancing transfection of nucleic acid in tissues and cells of the respiratory system.

With continued reference to FIG. 2, a dispersing apparatus may include a device comprising a compressed/pressurized inhalable aerosol delivery device and optionally equipped with a smart digital measurement capability for monitoring and ensuring optimal use of device, patient adherence and communicating patient use data to their healthcare provider. Such a device may include, such as but is not limited to, pulsating membrane nebulizers, vibrating mesh nebulizers, small volume nebulizers, pressured-metered dose inhalers, Smart dry powder inhalers and similar devices capable of inhaled delivery of dry powder or liquid/solution containing the particles of the invention. or. Such dispersing devices may have one or more separated medicine holding chambers containing the inhaled particles and configured to simultaneous or sequential administration of such particles according to a predetermined schedule as described in more detail below.

With continued reference to FIG. 2, in some embodiments, formulation comprising a transfection efficiency enhancing agent 104 and a therapeutic nucleic acid 108 may be administered by any administration route including but not limited to oral, intravenous, intramuscular, subcutaneous, Inhalation or Intranasal, Intratracheal, bronchial instillation and/or topical administration, transdermal, rectal, vaginal, topical, intrathecal, intra-vesicular, intra-tra-articular and the like.

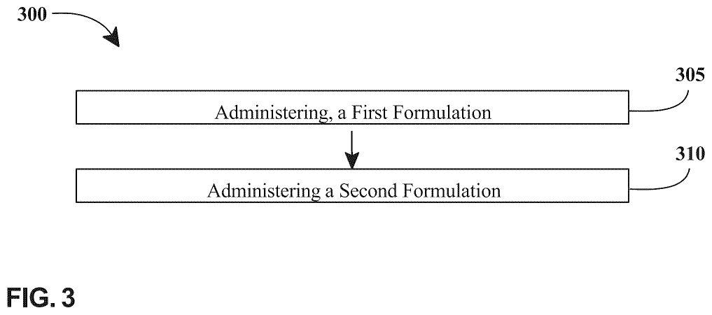

Referring now to FIG. 3, an exemplary embodiment of method 300 for enhanced delivery of nucleic acids to tissues and cells within the respiratory system is illustrated. At step 305, administration of a first formulation comprising a transfection efficiency enhancing agent 104 via Inhalation, Intranasal, Intratracheal, bronchial instillation, and/or topical administration to a subject in need thereof. Transfection efficiency enhancing agent 104 includes any transfection efficiency enhancing agent 104 as described herein. Inhalation, Intranasal, Intratracheal, bronchial instillation, and/or topical administration may include any delivery mechanism as described herein. In an embodiment, a time period may elapse before the administration of the second formulation occurs. A “time period,” as used in this disclosure, is any period of time that elapses between the administration of the first formulation and the second formulation to a subject in need thereof. For example, a first formulation containing transfection efficiency enhancing agent 104 may be administered via inhalation, intranasal, intratracheal, and/or direct bronchial instillation, a time period to, followed by, a second formulation containing therapeutic nucleic acid 108 in or attached to a delivery vehicle may be administered at a time after 5-60 minutes of the first formulation, via inhalation, intranasal, intratracheal, and/or direct bronchial instillation. In an embodiment, first formulation may be administered first in time, and second formulation may be administered second at a time after administration of first formulation.

With continued reference to FIG. 3, at step 310, administration of a second formulation comprising a therapeutic nucleic acid 108 in or attached to a delivery vehicle via Inhalation, Intranasal, Intratracheal, bronchial instillation, and/or topical administration route to the subject in need thereof. Therapeutic nucleic acid 108 includes any therapeutic nucleic acid 108 as described herein.

With continued reference to FIG. 3, this disclosure relates to methods, composition and formulations for improving pulmonary delivery of therapeutic nucleic acid 108, such as RNA, including messenger RNA (mRNA), mRNA vaccines, small interfering RNAs (siRNA), antisense oligonucleotides (ASO), aptamers, micro RNA (miRNA), transfer RNA (tRNA), Self-Amplifying RNA (saRNA), Gene Editing Components (e.g., CRISPR-Cas9), gene editing mRNA components, Guide RNA (gRNA) for CRISPR Systems, prime editing mRNA components, and circular RNA and DNA, and Plasmid DNA, to tissues and cells within the respiratory system. Delivery may be performed by any methodology described herein, including but not limited to inhalation, intranasal, bronchial instillation, topical administration route, and the like. This may involve enhancing transfection efficiency and, therefore, desired efficacy at low doses. This may be achieved by using an effective transfection efficiency enhancing agent 104 or composition 100 as a gateway drug or co-therapy. This co-therapy may include the administration of the inhalable solution via nebulization or dry-powder microsphere or nanosphere of the transfection efficiency enhancing agent 104 or compositions via dry powder inhaled device prior to (e.g., 5-60 minutes prior to) the administration of the therapeutic nucleic acid 108 in or attached to a delivery vehicle. or co-administration of the inhalable solution via nebulization or dry-powder microspheres of transfection efficiency enhancing agent 104 or compositions at the same time as the therapeutic nucleic acid in or attached to delivery vehicle. Exemplary transfection efficiency enhancing agent 104 or compositions may include 2-Mercaptoethane sulfonate, Cysteamine, and Vitamin C. Transfection efficiency enhancing agent 104 and compositions may be delivered in inhalable dry-powder microsphere form, solution form for nebulization, spray, gel or drops for intranasal or via any other method to the targeted site of action.

With continued reference to FIG. 3, in some embodiments, transfection efficiency enhancing agent 104 or compositions may be administered as gateway drug via Inhalation, Intranasal, Intratracheal, bronchial instillation and/or topical administration prior (e.g., 5-60 minutes prior) to administration of a therapeutic nucleic acid 108 in or attached to a delivery vehicle via inhalation or intranasal, Intratracheal, bronchial instillation and/or topical administration. A “gateway drug,” as used in this disclosure, is any formulation administered first, prior to the administration of a second formulation of therapeutic nucleic acid 108, to temporarily open the gate/barrier and facilitate entry of second formulation tissues and cells within the respiratory system. When a gateway drug is administered first, it may aid in enhancing the delivery, activity, and/or therapeutic effect, including efficacy and safety of the second formulation. For example, a formulation comprising a transfection efficiency enhancing agent 104 may be administered first to clear up mucus in airways or interact with membrane lipids so that therapeutic nucleic acid 108 can achieve better penetration into the cells of the respiratory system. In some embodiments the first formulation comprising a transfection efficiency enhancing agent 104 may be administered 5-60 minutes prior to administration of the second formulation comprising a therapeutic nucleic acid 108. In some embodiments, transfection efficiency enhancing agent 104 or compositions may be administered as co-therapy via inhalation, intranasal, intratracheal, bronchial instillation and/or topical administration together with a therapeutic nucleic acid 108 in or attached to a delivery vehicle.

With continued reference to FIG. 3, in some embodiments, a method of treating a pulmonary disease may include administering a transfection efficiency enhancing agent 104 in a single dose combination formulation also containing a therapeutic nucleic acid 108 via inhalation, Intranasal, Intratracheal, bronchial instillation and/or topical route of administration to a patient in need thereof. Transfection agents may facilitate the delivery of a higher percentage fraction of the total administered dose of the therapeutic nucleic acid 108 to cells within the respiratory system, such as, for example, lung tissue, therefore resulting in improved efficacy.

In some embodiments, transfection efficiency enhancing agent 104 or compositions may include a thiol in its active reduced form. For example, a transfection efficiency enhancing agent 104 may be selected from sodium 2-mercaptoethane sulfonate (HS-CH2-CH2-SO2-O—·Na+) or Cysteamine (HS-CH2-CH2-NH2), in therapeutically active form. In some embodiments, transfection efficiency enhancing agent 104 or compositions may include solutions or spray dried microspheres of sodium 2-mercaptoethane sulfonate. In some embodiments, transfection efficiency enhancing agent 104 or compositions may include solution or spray dried microspheres of Cysteamine. In some embodiments, when the nucleic acid is RNA in any form, the transfection efficiency enhancing agent 104 or compositions may include DNase, e.g., spray dried microsphere of DNase or solution of DNase for nebulization.

With continued reference to FIG. 3, in another embodiment, transfection efficiency enhancing agent 104 or compositions, e.g., MESNA or Cysteamine, may be used in a method of treating a pulmonary disease with or without a therapeutic nucleic acid 108.

With continued reference to FIG. 3, in another embodiment, a method for enhancing transfection into the respiratory system may include administering transfection efficiency enhancing agent 104 or compositions as co-therapy or a fixed-dose combination with a therapeutic nucleic acid 108 via Inhalation or intranasal, Intratracheal, bronchial instillation and/or topical administration.

With continued reference to FIG. 3, in some embodiments, a therapeutic nucleic acid 108 may be administered via Inhalation, Intranasal, Intratracheal, bronchial instillation and/or topical administration in a composition/and/or form including transfection efficiency enhancing agent 104 or compositions, e.g., comprising MESNA or Cysteamine, in a single combination formulation. In some embodiments, transfection efficiency enhancing agent 104 or compositions may be administered as gateway drug via inhalation, Intranasal, Intratracheal, bronchial instillation and/or topical administration prior (e.g., 5 to 60 minutes prior) to the administration of the therapeutic nucleic acid 108 via Intratracheal, bronchial instillation and/or topical administration. In some embodiments, transfection efficiency enhancing agent 104 or compositions may be administered as co-therapy via inhalation, Intranasal, Intratracheal, bronchial instillation, and/or topical administration together with a therapeutic nucleic acid 108.

With continued reference to FIG. 3, in some embodiments, The therapeutic Nucleic acid in a delivery vehicle 108 may suppress the expression of a defective gene involved in causing lung diseases such as Cystic Fibrosis and replace with the normal version of the gene with the regular protein expression for the treatment of Primary Ciliary Dyskinesia, Pulmonary Fibrosis, Pulmonary arterial hypertension (PAH), Lung cancer and other Lung and Airway diseases.

With continued reference to FIG. 3, in some embodiments, a therapeutic nucleic acid 108 may expressing normal version of the protein expressed by a defective or under-expressed gene, and a therapeutic nucleic acid 108 may provide gene replacement to a patient, e.g., a patient having a disease selected from cystic fibrosis caused by a defective CFTR gene; This section is going into specific which may limit the scope e.g. primary ciliary dyskinesia caused by defective DNAI1 gene; pulmonary arterial hypertension (PAH) caused by a defective or under-expressed bone morphogenetic protein type 2 receptor (BMPR2); sarco-endoplasmic reticulum calcium-ATPase 2a (SERCA2a); SIN3acomplex; endothelial nitric oxide synthase (ENOS); potassium voltage-gated channel, shaker-related subfamily, member 5 (KCNA5 or KV1.5); survivin; vasoactive intestinal peptide (VIP); or calcitonin gene-related peptide (CGRP); idiopathic pulmonary fibrosis (IPF) caused by defective or under-expressed sarco-endoplasmic reticulum calcium-ATPase 2a (SERCA2a); Caveolin-1; SMAD Family Member 7 (SMAD7); or Telomerase Reverse Transcriptase (TERT).

With continued reference to FIG. 3, in some embodiments, a therapeutic nucleic acid 108 may modulate expressions a cytokine that cause inflammation, e.g., IL-4, IFN-β, or IL-10; e.g., Xalud's, XT-150, a DNA plasmid that expresses a modified form of IL-10, e.g., administered to a patient suffering from an inflammatory disease, e.g., asthma or COPD. In some embodiments, a therapeutic nucleic acid 108 may encode a tumor suppressor gene product, e.g., p53 or tumor suppressor candidate 2 (TUSC2), e.g., administered to a patient suffering from lung cancer, e.g., non-small cell lung cancer.

With continued reference to FIG. 3, in some embodiments, a method of treating cystic fibrosis may include administering a spray-dried microsphere of transfection efficiency enhancing agent 104 or compositions may comprise MESNA or Cysteamine, DSPC, and CaCl2 (e.g., in a dosage of 5-100 mg MESNA or Cysteamine), via Inhalation, Intranasal, Intratracheal, bronchial instillation and/or topical administration Inhalation, to a subject in need thereof. Followed by administration of mRNA encoding normal CFTR, e.g., at a dosage of 5 mg-25 mg, e.g., 5-60 minutes later, via Inhalation or Intranasal, Intratracheal, bronchial instillation and/or topical administration.

With continued reference to FIG. 3, in some embodiments, a method of treating primary ciliary dyskinesia may include administering a spray-dried microsphere formulation comprising MESNA or Cysteamine, DSPC, and CaCl2 (e.g., in a dosage of 5-100 mg MESNA or Cysteamine), via Inhalation, Intranasal, Intratracheal, bronchial instillation and/or topical, followed by administration of mRNA encoding the corrected gene, 5-60 minutes via Inhalation or Intranasal, Intratracheal, bronchial instillation and/or topical administration e.g., In some embodiments, formulation of DNAH5 mRNA in a delivery vehicle such as inhalable lipid nanoparticles may be used.

With continued reference to FIG. 3, in some embodiments, a composition and/or method described herein may be used to treat a pulmonary disease associated with a genetic defect, e.g., cystic fibrosis caused by a defective CFTR gene or primary ciliary dyskinesia caused by multiple defective genes, e.g., a defective DNAI1, DNAH5, or DNAH11. In some embodiments, a therapeutic nucleic acid 108 may include mRNA expressing a normal version of a protein expressed by a defective gene, e.g., wherein the pulmonary disease is cystic fibrosis caused by a defective CFTR gene and the therapeutic nucleic acid 108 is mRNA expressing normal functional CFTR.

With continued reference to FIG. 3, In some embodiments, a pulmonary disease may be associated with a defective, under-expressed or over-expressed gene, and a therapeutic nucleic acid 108 may code for expressing a therapeutically effective level of a normal protein to replace or supplement a protein expressed by a defective or under-expressed gene. Compositions and/or methods described herein may be used to treat, for example, cystic fibrosis caused by a defective CFTR gene; primary ciliary dyskinesia caused by a defective DNAI1 gene; pulmonary arterial hypertension (PAH) caused by a defective or under-expressed bone morphogenetic protein type 2 receptor (BMPR2); sarco-endoplasmic reticulum calcium-ATPase 2a (SERCA2a); SIN3a complex; endothelial nitric oxide synthase (ENOS); potassium voltage-gated channel, shaker-related subfamily, member 5 (KCNA5 or KV1.5); surviving; vasoactive intestinal peptide (VIP); or calcitonin gene-related peptide (CGRP); idiopathic pulmonary fibrosis (IPF) caused by defective or under-expressed sarco-endoplasmic reticulum calcium-ATPase 2a (SERCA2a); caveolin-1; SMAD Family Member 7 (SMAD7); or telomerase reverse transcriptase (TERT).

With continued reference to FIG. 3, in some embodiments, a disease which may be treated by a composition and/or method described herein may include an inflammatory disease, e.g., asthma or COPD, for example, using a therapeutic nucleic acid 108 encoding a cytokine that modulates or reduces inflammation, e.g., IL-4, IFN-β, or IL-10; e.g. wherein the nucleic acid is Xelus's, XT-150, a DNA plasmid that expresses a modified form of IL-10.

With continued reference to FIG. 3, in some embodiments, a disease which may be treated by a composition and/or method described herein may include lung cancer, e.g., non-small cell lung cancer or small cell lung cancer. For example, a therapeutic nucleic acid 108 may encode a tumor suppressor gene product, e.g., p53 or tumor suppressor candidate 2 (TUSC2).

With continued reference to FIG. 3, In some embodiments, a method of treating cystic fibrosis may include administering a spray-dried microsphere formulation comprising MESNA or Cysteamine, DSPC, and CaCl2 in a dosage of 10-150 mg via Inhalation, Intranasal, Intratracheal, bronchial instillation and/or topical administration as gateway drug at a dosage of 5-100 mg, followed by administration of mRNA encoding correct CFTR, e.g 10-60 minutes after administration of transfection enhancing agent via Inhalation or Intranasal, Intratracheal, bronchial instillation and/or topical administration.

With continued reference to FIG. 3, in some embodiments, a method of treating primary ciliary dyskinesia may include administering a spray-dried microsphere formulation comprising MESNA or Cysteamine, DSPC, and CaCl2 in a dosage of 5-150 mg via Inhalation, Intranasal, Intratracheal, bronchial instillation and/or topical administration as gateway drug, and 5-60 minutes later followed by administration, of mRNA encoding a corrected protein, e.g., encoding DNAI1, DNAH5, or DNAH11, via Inhalation or Intranasal, Intratracheal, bronchial instillation and/or topical administration.

With reference to FIGS. 1. 2. and 3, a list of conditions and diseases that may benefit from the present invention, including any composition, agent, and/or method as described herein in reference to FIGS. 1-7 may include:
Primary ciliary dyskinesia (PCD)
Cystic Fibrosis (CF) Pulmonary arterial hypertension (PAH)
Idiopathic pulmonary fibrosis (IPF)
Bronchiectasis (BE)
Acute Respiratory Distress Syndrome (ARDS)
Chronic Obstructive Pulmonary Disease (COPD)
Alpha-1 Antitrypsin Deficiency (AATD)
Sinusitis
Rhinosinusitis
Bronchiolitis Obliterans (BO)
Pulmonary Sarcoidosis
Emphysema
Bronchitis
Pneumonia
Pneumonitis
Asthma
Lung cancer
Non-Small Cell Lung Cancer (NSCLC)
Other lung and airway diseases
Other muco-obstructive diseases
Smith-Golabi-Behmel Syndrome (SGS)
Other cardiopulmonary diseases
Tuberculosis (TB)
Viral, fungal and bacterial infections of organs in the respiratory and other vital systems in the human body e.g. Ear infection, Respiratory Syncytial Virus (RSV) Infection
Other genetic diseases
Autoimmune and Inflammatory Diseases
Neurological and Neurodegenerative Diseases
Blood Disorders

In some embodiments, cells to be transfected are respiratory epithelial cells, alveolar cells including Type 1 alveolar cells and Type 2 alveolar cells, ciliated epithelial cells, goblet cells, endothelial cells, macrophages, basal cells, club cells, mast cell, dendritic cells, brush cells, neutrophils, eosinophils and/or neuroendocrine cells. In some embodiments, transfection efficiency enhancing agent 104 or compositions, e.g., MESNA or Cysteamine may be used in a method of enhancing transfection of nucleic acid in tissues and cell of respiratory system.

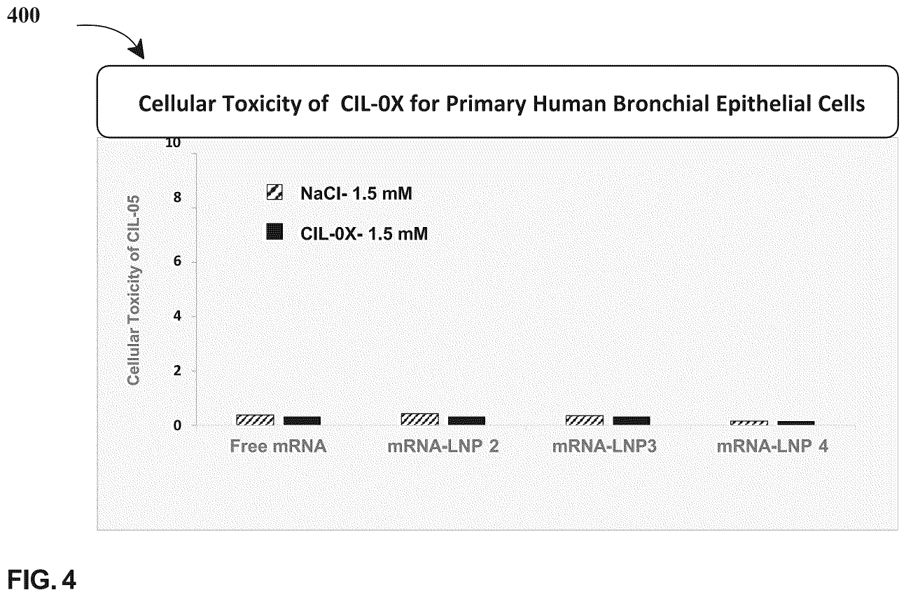

Referring now to FIG. 4, an exemplary embodiment 400 of cellular toxicity data of composition 100 comprising of Transfection enhancing agent and nucleic acid in lipid nanoparticles delivery vehicles tested in primary human respiratory epithelial cells matured at Air Liquid interface to mimic in vivo lung-like conditions is illustrated. Cellular toxicity for primary human respiratory epithelial cells of composition 100 is illustrated as demonstrated in comparison with a control arm consisting of 1.5 mM sodium chloride versus 1.5 mM Transfection enhancing agent 104 and nanoparticles carrying mRNA 108. Toxicity was low, and there was no statistically significant difference in toxicity between the 1.5 mM sodium chloride control arm and 1.5 mM Transfection enhancing agent (CIL-0X), and free mRNA was noted. Furthermore, no statistically significant difference in toxicity between the 1.5 mM sodium chloride control arm and 1.5 mM transfection enhancing agent (CIL-0X) arm with mRNA-LNPs was noted.

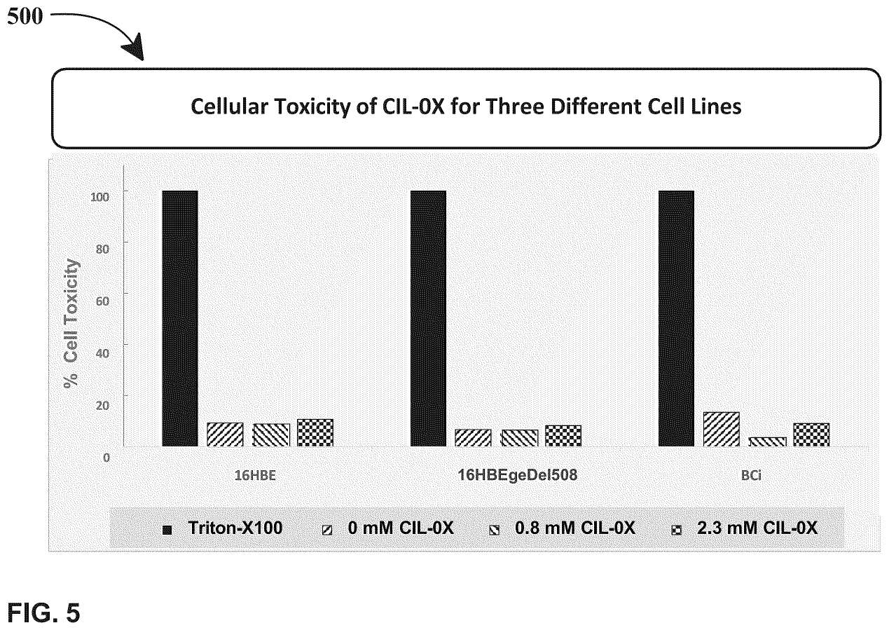

Referring now to FIG. 5, an exemplary embodiment 500 illustrates the percentage of cellular toxicity/safety of composition 104 in various human immortalized respiratory epithelial cell lines. Various compositions of 104 with varying concentrations of transfection enhancing agents, CIL-0X were tested in three different cell lines. The first cell line 16HBE14o-(16HBE) is a bronchial epithelial cell line. These cells were derived from normal human bronchial epithelial tissue and were immortalized using a viral gene, the SV40 large T antigen. Various strengths of transfection enhancing agent and composition 104, including 0 mM CIL-0X, 0.8 mM CIL-0X, and 2.3 mM CIL-0X, were tested for toxicity in the 16HBE cell line and Triton-X100 as a positive control. The various strengths of composition 100 all achieved less than 20% cellular toxicity in epithelial cell line. The second cell line CFF are 16HBE cells gene-edited and expressing mutant Fdel508 CFTR modeled for Cystic Fibrosis research. Various strengths of composition 104, including 0 mM CIL-0X, 0.8 mM CIL-0X, and 2.3 mM CIL-0X, were tested for toxicity in this cell line, along with Triton-X100 as a positive control. All composition 104 with varying concentrations of transfection enhancing agent (CIL-0X) including 0 mM CIL-0X, 0.8 mM CIL-0X, and 2.3 mM CIL-0X demonstrated less than 15% cellular toxicity of Triton X100. The third cell line BCi-NS1.1 (BCi) is an immortalized human respiratory epithelial cell line demonstrating abnormal ciliary beating pattern and microtubule defects as is seen in some mutations causing Primary Ciliary Dyskinesia. Various composition 100 including 0 mM CIL-0X, 0.8 mM CIL-0X, and 2.3 mM transfection enhancing agents CIL-0X were tested for toxicity in the BCi cell line along with Triton-X100 as positive control. The various strengths of the all composition of transfection enhancing agent 104, all achieved less than 15% cellular toxicity in basal cell line. This data demonstrates that transfection enhancing agent and composition 104 is safe for airway cells from healthy donors and cells with the most common Cystic Fibrosis mutation or dysfunctional cilia as seen in Primary Ciliary Dyskinesia.

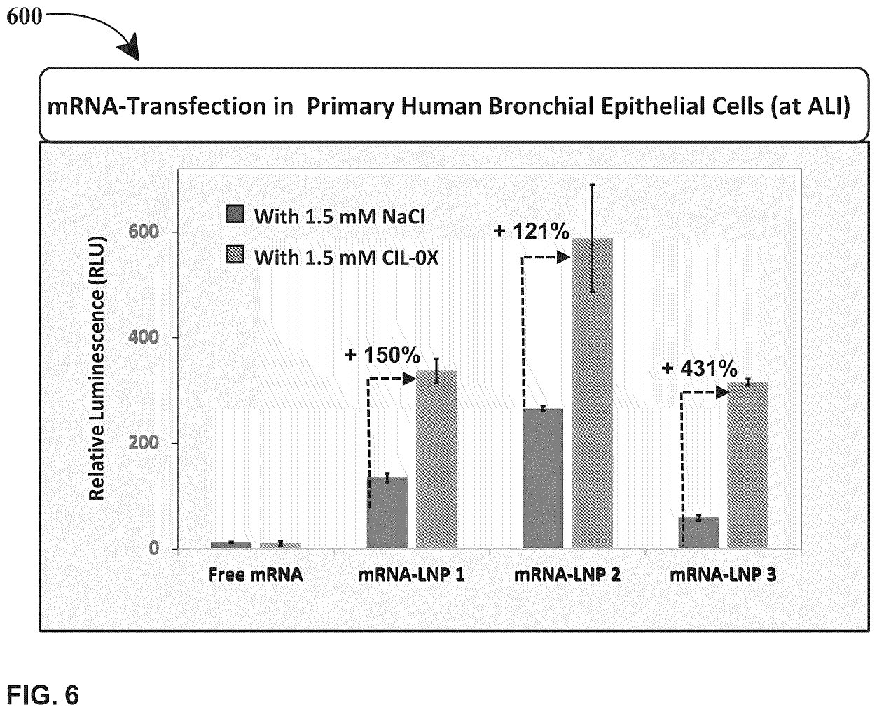

Referring now to FIG. 6, an exemplary embodiment 600 illustrates the impact of CIL-0X (transfection enhancing composition) on the delivery of Luciferase mRNA-in lipid nanoparticle delivery vehicle to Primary human bronchial epithelial cells matured at Air-Liquid Interface. Luciferase mRNA was prepared in 4 different forms, including free mRNA without a delivery vehicle and in three different lipid nanoparticle vehicles, i.c., mRNA-LNP-1, mRNA LNP-2, mRNA-LNP3. The expression of Luciferase was assessed by measuring Luciferase enzyme activity (using Luciferase Reporter Assay), after 24 hours of nebulized administration of free mRNA and mRNA in LNPs vehicles to the primary human epithelial cells that were pre-treated with either 1.5 mM CIL-0X (transfection enhancing agent/composition) as the TEST arm or 1.5 mM NaCl as the CONTROL arm, 30 minutes before mRNA-LNP administration. The activity of expressed bioluminescent luciferase enzymes was used to quantify the difference in mRNA transfection between free mRNA and mRNA-LNPs in primary human epithelial cells in both the TEST (CIL-0X) arm and CONTROL (NaCl) arm. As shown in FIG. 6, Free mRNA administration resulted in little, or no luciferase activity expressed in cells. All mRNA-LNP formulations resulted in increased luciferase expression compared to free mRNA. Luciferase expression was significantly enhanced in the TEST arm of cells that were pretreated with 1.5 mM CIL-OX compared to cells in CONTROL arm pretreated with 1.5 mM saline control. Specifically, the Luciferase expression was found to have increased by 150% for mRNA-LNP1, 121% for mRNA-LNP2, and 431% for mRNA-LNP3. These results demonstrate the statistically significant increase/enhancement in transfection efficiency of mRNA in cells treated with 1.5 mM CIL-0X versus the control arm.

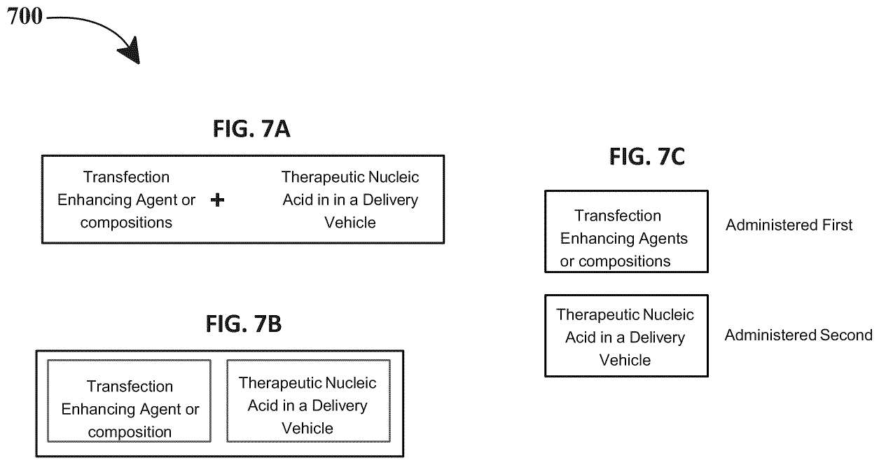

Referring now to FIG. 7, an exemplary embodiment 700 of various methods of improving transfection efficiency of nucleic acid in or attached to a delivery vehicle are illustrated. Referring first to FIG. 7A, transfection enhancing agent 104 may be delivered to the airway along with therapeutic nucleic acid 108 in a single formulation as a fixed-dose combination via inhalation, intranasal, intratracheal, direct instillation, and/or topical administration. Referring now to FIG. 7B, transfection enhancing agent 104 may be delivered simultaneously along with nucleic acid whereby transfection enhancing agent 104 and therapeutic nucleic acid 108 are formulated separately in separate dose and their respective vehicle. The two formulations are administered simultaneously but as separate doses to the cells of the respiratory system. Delivery vehicles may include any delivery vehicle as described above in more detail in reference to FIGS. 1-6. Referring now to FIG. 7C, transfection enhancing agent 104 may be administered first, whereby a time period may pass, and then therapeutic nucleic acid 108 in or attached to a delivery vehicle may be administered second. FIGS. 7A-7C may be implemented using any methodology and/or composition described above in reference to FIGS. 1-6.

It is contemplated that any embodiment discussed in this specification can be implemented with respect to any method of the invention and vice versa. It will also be understood that specific embodiments described herein are shown as a way of illustration and not as limitations of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. Incorporation by reference is limited such that no subject matter is incorporated that is contrary to the explicit disclosure herein, no claims included in the documents are incorporated by reference herein, and any definitions provided in the documents are not incorporated by reference herein unless expressly included herein.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) or “component of” are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the pre-ceding discussion, a numerical value herein that is modified by a word of approximation such as “about” may vary from the stated value by at least ±1, 2, 3, 4, 5, 6, 7, 10, 12, 15, 20 or 25%.

All of the devices and/or methods disclosed and claimed herein can be made and executed without undue experimentation in light of the present disclosure. While the devices and methods of this invention have been described in terms of preferred embodiments, it will be apparent to those of skill in the art that variations may be applied to the devices and/or methods and in the steps or in the sequence of steps of the method described herein without department form the concept, spirit and scope of the invention. All such similar substitutes and modifications apparent to those skilled in the art are deemed to be within the spirit, scope and concept of the invention as defined by the appended claims.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and compositions according to the present disclosure. Accordingly, this description is meant to be taken only by way of example, and not to otherwise limit the scope of this invention.

Exemplary embodiments have been disclosed above and illustrated in the accompanying drawings, FIG. 1-7. It will be understood by those skilled in the art that various changes, omissions and additions may be made to that which is specifically disclosed herein without departing from the spirit and scope of the present invention.

What is claimed is:

1. A composition for enhanced delivery of nucleic acids to lung and airway cells for preventing and treating pulmonary diseases comprising of:
at least a transfection efficiency enhancing agent; and
a therapeutic nucleic acid with a delivery vehicle.

2. The composition of claim 1, wherein the composition is in a solution form for nebulization, nasal spray or dropper.

3. The composition of claim 1, wherein the composition is in a dry powder form for delivery via an inhaler.

4. The composition of claim 1, wherein the composition is in a gel form for delivery via topical administration.

5. The composition of claim 1, wherein the therapeutic nucleic acid further comprises a therapeutic nucleic acid.

6. The composition of claim 1, wherein the therapeutic nucleic acid further comprises a prophylactic nucleic acid.

7. The composition of claim 1, wherein the transfection efficiency enhancing agent may further comprise a thiol compound in its active reduced form.

8. The composition of claim 1, wherein the transfection efficiency enhancing agent comprising of one or more agents selected from a group of compounds consisting of: sodium 2-mercaptoethane sulfonate (HS-CH2-CH2-SO2-O—·Na+) or cysteamine (HS-CH2-CH2-NH2), in reduced (thiol) form, ascorbic Acid or its salts, DNase, Bromohexine, Reduced Glutathione, N-derivatives and C-derivates of amino acid cysteine, Di-peptide of cysteine and glutamic acid, Di-peptide of aspartic acid, Aspartic Acid Salts N-Acetylecysteine, N-Butylecysteine, Ambroxol hydrochloride, Vitamin E and other compounds.

9. The composition of claim 8, further comprising a lipid and an additional agent.

10. The composition of claim 1, wherein the therapeutic nucleic acid is RNA selected from a group of compounds consisting of: RNA, including messenger RNA (mRNA), mRNA vaccines, small interfering RNAs (siRNA), antisense oligonucleotides (ASO), aptamers, micro RNA (miRNA), transfer RNA (tRNA), Self-Amplifying RNA (saRNA), Gene Editing Components (e.g., CRISPR-Cas9), gene editing mRNA components, Guide RNA (gRNA) for CRISPR Systems, prime editing mRNA components, and circular RNA.

11. The composition of claim 1, wherein the therapeutic nucleic acid is selected from the group of compounds consisting of: DNA, Plasmid DNA, minicircle DNA, mitochondrial DNA, synthetic oligonucleotide, linear DNA (for CRISPR/Cas9 gene editing or CAR-T cell production).

12. The composition of claim 1, wherein the therapeutic nucleic acid is comprised of a delivery vehicle selected from the group consisting of: a Nanoparticle (LNP), a Viral Vector, a Polymeric Nanoparticle, an Exosome or extracellular vesicle, a peptide-based vehicle, a gold nanoparticle, a cell-penetrating peptides and a dendrimer.

13. The composition of claim 1, wherein the composition is in a dry powder microsphere nanospheres or nanoparticle form.

14. A method of enhanced delivery of therapeutic nucleic acids to cells of respiratory system for the treatment or prevention of pulmonary diseases, the method comprising administering a formulation comprising a transfection efficiency enhancing agent and a therapeutic nucleic acid attached to a delivery vehicle via inhalation, to a subject in need thereof and wherein the transfection efficiency enhancing agent enhances the delivery of the therapeutic nucleic acid to cells of the respiratory system.

15. A method of enhanced delivery of nucleic acids to cells of the respiratory system the method comprising administering a first formulation comprising a transfection efficiency enhancing agent via inhalation, to a subject in need thereof and administering a second formulation comprising a therapeutic nucleic acid attached to a delivery vehicle via inhalation to the subject in need thereof.

16. The method of claim 15, wherein the first formulation is administered 5 to 60 minutes before the second formulation is administered.

17. The method of claim 15, wherein the transfection efficiency enhancing agent comprises sodium 2-mercaptoethane sulfonate. (MESNA).

18. The method of claim 15, wherein the transfection efficiency enhancing agent comprises 2-Aminoethane-1-thiol. (Cysteamine).

19. The method of claim 15, wherein the therapeutic nucleic acid comprises RNA or DNA.

20. The method of claim 19, wherein the therapeutic nucleic acid is selected from the group consisting of: DNA, Plasmid DNA, minicircle DNA, mitochondrial DNA, synthetic oligonucleotide, linear DNA (for CRISPR/Cas9 gene editing), for gene addition or gene-editing.

21. The method of claim 19, wherein the therapeutic nucleic acid is selected from the group consisting of: RNA, including messenger RNA (mRNA), mRNA vaccines, small interfering RNAs (siRNA), antisense oligonucleotides (ASO), aptamers, micro RNA (miRNA), transfer RNA (tRNA), Self-Amplifying RNA (saRNA), Gene Editing Components (e.g., CRISPR-Cas9), gene editing mRNA components, Guide RNA (gRNA) for CRISPR Systems, prime editing mRNA components, and circular RNA. comprises mRNA, miRNA, siRNA, and antisense oligonucleotide (ASO)], and RNA aptamers.

22. The method of claim 15, wherein the therapeutic nucleic acid is a component of a vaccine.

23. The method of claim 15, wherein the therapeutic nucleic acid is a component of a therapeutic.
